(19) United States
(12) Patent Application Publication
(10) Pub. No.: US 2026/0102534 A1
(43) Pub. Date: Apr. 16, 2026

(54) EXPOSED WASTE ODOR NEUTRALIZATION

(71) Applicant: ExWON, LLC, Castle Rock, CO (US)

(72) Inventors: Mark Adams, Castle Rock, CO (US); David M. Payne, Utica, NE (US)

(21) Appl. No.: 19/287,513

(22) Filed: Jul. 31, 2025

Related U.S. Application Data
(60) Provisional application No. 63/705,572, filed on Oct. 10, 2024.

(51) Int. Cl. A61L 9/14

(52) U.S. Cl. CPC A61L 9/14; A61L 2209/134

(57) ABSTRACT

A waste-handling facility odor mitigation system includes mobile waste-handling equipment, an odor-mitigating solution tank, a compressor and at least one nozzle. The waste-handling equipment has at least one radiator fan and at least one waste-handling blade or bucket. The odor-mitigating solution tank is mounted to the waste-handling equipment. The compressor is configured to pressurize ambient air. The at least one nozzle is configured to discharge a mixture of the odor-mitigating solution and the compressed air.

### CROSS REFERENCE TO RELATED APPLICATIONS

This application claims the priority benefit of U.S. Provisional Application No. US 63/705,572 filed 10 Oct. 2024, pending, which is incorporated herein by reference in its entirety.

### SUMMARY

The disclosure describes an exposed waste odor neutralizing system. The exposed waste odor neutralizing system inlcudes mobile waste-handling equipment, an odor-mitigating solution tank mounted to the waste-handling equipment, a compressor configured to pressurize ambient air and at least one nozzle. The mobile waste-handling equipment includes at least one waste-handling effector. The nozzle is configured to discharge a mixture of the pressurized ambient air and odor-mitigating solution from the odor-mitigating solution tank.

The disclosure also describes an odor neutralizer dispersal system. The odor neutralizer dispersal system includes an odor-mitigating solution tank, a compressor and at least one nozzle. The odor-mitigating solution tank is configured for mounting to mobile waste-handling equipment which includes at least one waste-handling blade or bucket. The compressor is also configured for mounting to the waste-handling equipment and for pressurizing ambient air. The a least one nozzle is configured for mounting to the waste-handling equipment and for discharging a mixture of the odor-mitigating solution and the pressurized ambient air.

Still further, the disclosure describes a method for mitigating waste-handling facility odor. The method includes, at the working area of a waste-handling facility, manipulating waste with a blade or bucket of a mobile waste-handling and, contemporaneously with the manipulating, discharging a mixture of odor-mitigating solution and compressed air.

### BRIEF DESCRIPTION OF THE FIGURES

The summary above, as well as the following detailed description of illustrative embodiments, is better understood when read in conjunction with the appended drawings. For the purpose of illustrating the disclosure, example constructions of the disclosure are shown in the drawings. However, the disclosure is not limited to specific methods and instrumentalities disclosed herein. Moreover, those in the art will understand that the drawings are not to scale. Wherever possible, like elements have been indicated by identical numbers.

Embodiments of the disclosure will now be described, by way of example only, with reference to the following diagrams wherein:

FIG. 1 schematically illustrates components of an example exposed waste odor neutralizing system.

FIG. 2 illustrates an external front view of an example odor neutralizer dispersal system.

FIG. 3 illustrates an internal side view of the odor neutralizer dispersal system of FIG. 2.

FIG. 4 illustrates a front view of a radiator grill with odor-mitigation solution nozzles coupled therewith.

FIG. 5 illustrates a perspective view of an example nozzle suitable for use in association with disclosed system and methods.

FIG. 6 illustrates an example exposed waste odor neutralizing system in use during waste manipulation.

### DETAILED DESCRIPTION

The following detailed description illustrates embodiments of the disclosure and manners by which they can be implemented. Although the best mode of carrying out the disclosure has been disclosed, those skilled in the art would recognize that other embodiments for carrying out or practicing the disclosure are also possible.

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

Solid waste facilities or waste-handling facilities such as landfills and transfer stations have been widely known to cause odor issues to residents, communities and businesses in the vicinity of these facilities. These odors adversely impact nearby communities and have resulted in a multitude of lawsuits against owner/operators. Mitigating the odors eliminates the nuisance issue and reduces liability exposure for these facilities.

Odor-mitigating solution dispersal systems are known for use on waste-handling facilities but are typically mounted on stationary objects, such as fences, overhead misters in transfer stations, and trailers, which are situated some distance from the waste handling area. Other odor-mitigating solution dispersal systems have been mounted on vehicles whose function is limited to the transport of the solution dispersal systems to various locations at a waste-handling facility. Such vehicles risk impeding the efficient and safe operation of the waste-handling facility where the vehicles are close to the active operating area for any period of time.

Disclosed waste-handling facility odor mitigation systems, methods for mitigating waste-handling facility odor and methods for assembling waste-handling facility odor mitigation systems target a primary source of the odors—the active operating area of solid waste facilities where waste is directly exposed to the environment as it is being handled by heavy, waste-processing machinery and/or waste-handling equipment. Disclosed advancements disperse odor-mitigating solutions and/or chemicals over the exposed waste continuously, near-continuously or periodically over the course of the operating day to mitigate odors emanating from discarded materials such as solid waste.

Additional aspects, advantages, features, and objects of the disclosure will be made apparent from the drawings and the detailed description of the illustrative embodiments construed in conjunction with the appended claims that follow.

It will be appreciated that features of the disclosure are susceptible to being combined in various combinations without departing from the scope of the disclosure as defined by the appended claims.

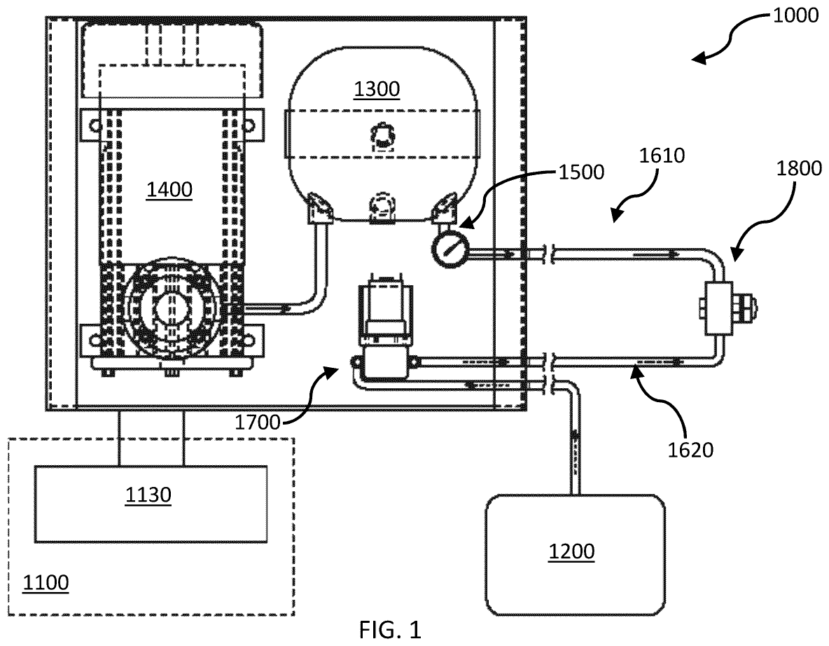

FIG. 1 schematically illustrates an example exposed waste odor neutralizing system 1000 which may also be considered a waste-handling facility odor mitigation system. Exposed waste odor neutralizing system 1000 includes waste-handling equipment 1100, an odor-mitigating solution tank 1200, a compressor tank 1300, a compressor 1400 and one or more nozzles 1800.

Odor-mitigating solution tank 1200 is configured to contain a solution, chemical or chemicals for interacting with odor-causing vapors emitted from solid waste. In an example, odor-mitigating solution tank 1200 contains QuickAirV™ and is small enough to be easily handled by facility personnel and fit on the waste-handling equipment while having sufficient volume to not require refill during a waste-handling shift. Solution tank regulating pump 1700 is coupled with odor-mitigating solution tank 1200 to control flow of odor-mitigating solution either manually or remotely. A motor of the solution tank regulating pump 1700 may be electrically coupled with an electrical output 1130 of the waste-handling equipment 1100 to energize the motor. A solution hose 1620 may be coupled between solution tank regulating pump 1700 and nozzles 1800 for communicating odor-mitigating solution therebetween.

Compressor 1400 is configured to pressurize ambient air to compressor tank 1300 which is configured to store pressurized ambient air from compressor 1400 for eventual release to one or more nozzles 1800. Exposed waste odor neutralizing system 1000 may further include a flow regulator 1500 coupled with compressor tank 1300 to control flow of pressurized ambient air therefrom. An air hose 1610 coupled with flow regulator 1500 is configured to communicate pressurized air between compressor tank 1300 and nozzles 1800. Flow regulator 1500 may be configured for manual or remote adjustment.

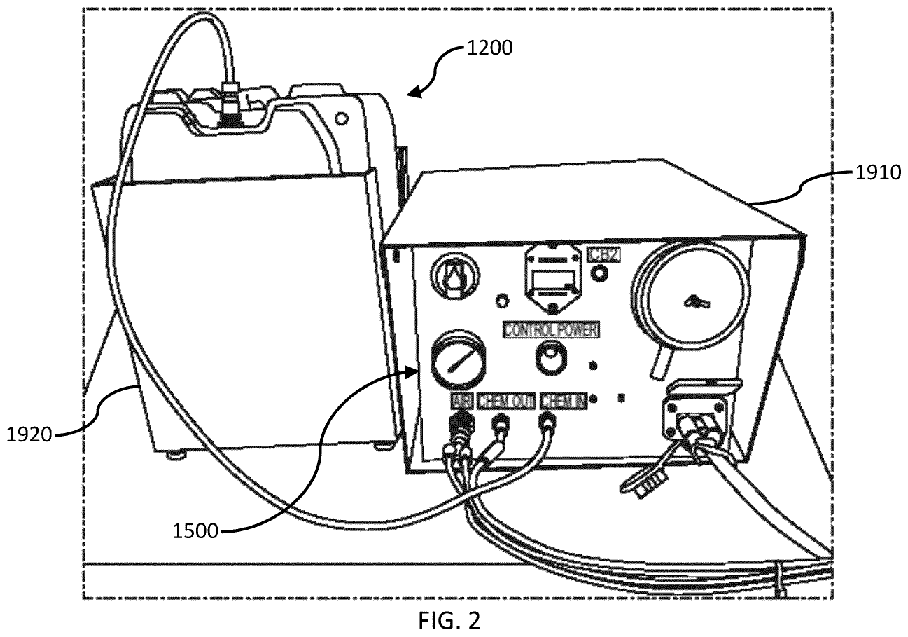

Referring to FIGS. 2 & 3, various components of exposed waste odor neutralizing system 1000 may be contained or partially contained in one or more housings 1910, 1920 to provide an odor neutralizer dispersal system as a possible subsystem of disclosed exposed waste odor neutralizing system 1000. Housings may facilitate mounting of the subsystem to a support surface such as a surface of mobile waste-handing equipment 1100 and may further include one or more electrical, mechanical or fluid interfaces or couplings, wiring harnesses, ports or valves to support interaction with waste-handling equipment and/or between system components.

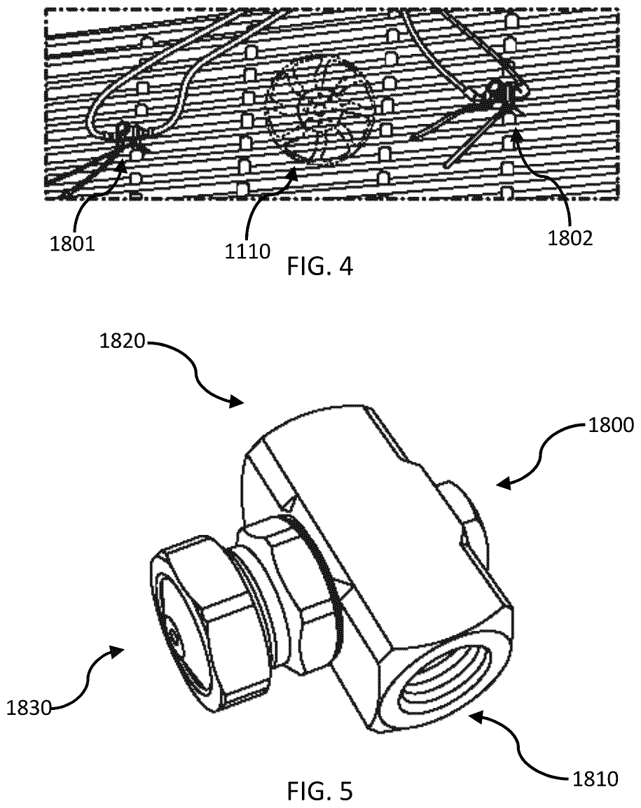

Returning to FIG. 1, nozzles 1800 are configured to discharge a mixture of odor-mitigating solution and compressed air. In an example, nozzles 1800 are configured to discharge the mixture downstream of a waste-handling equipment radiator fan. In a further example, nozzles 1800 are mounted to waste-handling equipment 1100 downstream of waste-handling equipment radiator fan 1110. For example, referring to FIG. 4, nozzles 1801 and 1802 may be mounted to a grill of waste-handling equipment radiator fan 1110. In another example, nozzles 1800 may be mounted to waste-handling equipment 1100 without consideration for the position or presence of radiator fan 1110 and rely upon pressure or motion of the waste-handling equipment for solution distribution and/or dispersal.

Embodiments may include any number of nozzles 1800 sufficient to provide the required or desired volume of odor-mitigating solution. While FIG. 4 shows two nozzles 1801 and 1802, disclosed systems may include a greater or lesser number than this. In another example, one or more nozzles 1800 may be mounted downstream of one or more supplemental fans in addition to or alternatively to radiator fan 1110. The supplemental fans may rotate at a fixed angle or may pivot or oscillate while rotating.

Referring to FIG. 5, each nozzle 1800 may further include an output 1830, a first input 1810 in fluid communication with output 1830 and a second input 1820 also in fluid communication with output 1830. First input 1810 is configured for coupling with odor-mitigating solution tank 1200, for example, through regulating pump 1700 and solution hose 1620 and second input 1820 is configured for coupling with compressor 1400, for example, by an air hose 1620. Output 1830 is configured for emitting or discharging a mixture of odor-mitigating solution received through input 1810.

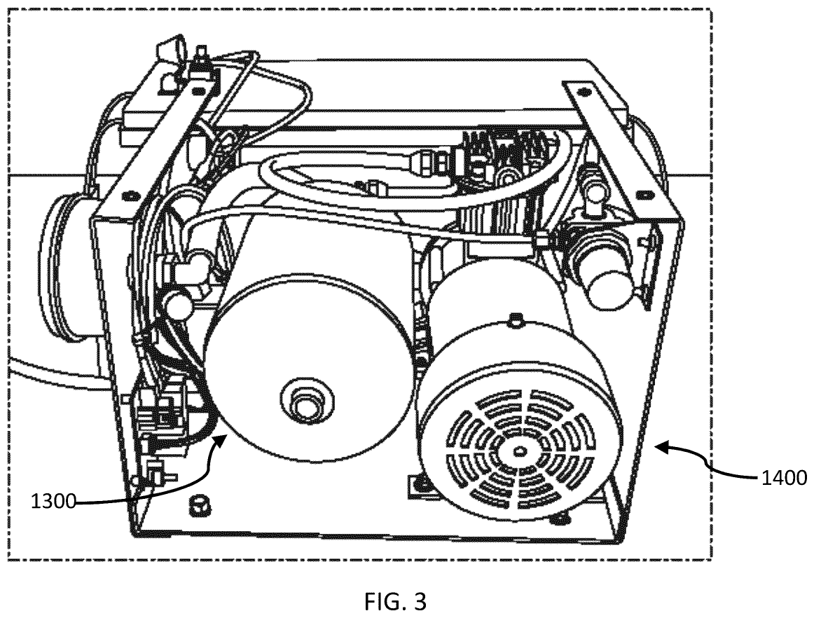

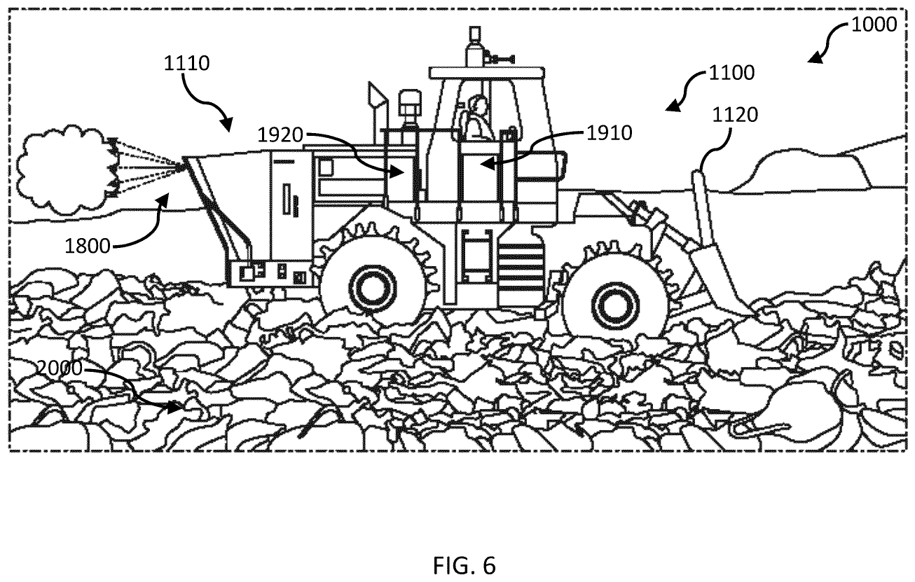

Odor neutralizer dispersal systems described with reference to FIGS. 2-5 may be suitable for use in any of a variety of exposed waste odor neutralizing systems and/or waste-handling facility odor mitigation systems. FIG. 6 illustrates an example exposed waste odor neutralizing system 1000 which may be an implementation of system 1000 of FIG. 1. Exposed waste odor neutralizing system 1000 includes waste-handling equipment 1100 and an odor neutralizer dispersal system including nozzle(s) 1800 as well as other components described with reference to FIGS. 1-5 and partially or wholly contained within housings 1910 and 1920. The odor neutralizer dispersal system, as contained or partially contained within housings 1910 and 1920, may take any of a variety of dimensions suitable for mounting to one or more surfaces of waste-handling equipment 1100. In an example, housing 1910 is approximately 35 in. long, approximately 20 in. wide and approximately 15 in. high while housing 1910 is approximately 35 in. high, approximately 20 in. wide and approximately 15 in. long.

Waste-handling equipment 1100, which is engaged in the daily management of exposed waste, may be implemented as one or more waste-processing, waste-handling or waste-managing machines or vehicles. The waste-handling equipment may be mobile as with a terrestrial vehicle or an aerial vehicle configured to travel on or over the surface of a landfill or other waste-processing facility. In an example, the waste-handling equipment includes at least one wheel, track or tread. Mobile waste-handling equipment 1100 may include at least one radiator fan 1110 and a waste-handling effector or tool such as a bucket or blade 1120.

Disclosed odor neutralizer dispersal systems, exposed waste odor neutralizing systems and waste-handling facility odor mitigation systems may be suitable for assembly according to any of a variety of methods. An example method includes providing a mobile waste-handling machine including at least one waste-handling blade or bucket, mounting an odor-mitigating solution tank to the machine and mounting a compressor tank to the machine. A compressor configured to pressurize ambient air to the compressor tank is coupled to an input of the compressor tank.

A flow regulator configured to control flow of compressed air from the compressor tank is coupled to an output of the compressor tank. A solution tank regulating pump configured to supply odor-mitigating solution from the odor-mitigating solution tank is coupled to an output of the odor-mitigating solution tank and a motor of the solution tank regulating pump is coupled to an electrical energy source of the machine. The flow regulator and solution tank regulating pump may be configured for local, manual adjustment or for remote adjustment.

At least one nozzle is coupled with the solution tank regulating pump by a solution hose and to the compressor tank by the air hose. The nozzle or nozzles may then be provided downstream of a radiator fan of the machine. The nozzles may be mounted to a radiator grill, for example, at a louver.

Disclosed odor neutralizer dispersal systems, exposed waste odor neutralizing systems and waste-handling facility odor mitigation systems may be suitable for use according to any of a variety of methods. One possible method includes, at the working face of a waste-handling facility, manipulating waste with a blade or bucket of a mobile waste-handling machine having at least one rotating radiator fan and, simultaneously, contemporaneously, periodically or intermittently with the manipulating, discharging a mixture of odor-mitigating solution and compressed air downstream of the rotating radiator fan so that the odor-mitigating solution and/or chemical is continuously, near-continuously or periodically dispersed over the exposed waste during the operating day. Discharging the mixture may further include discharging through an output of a nozzle.

The odor-mitigating solution may be pumped from an odor-mitigating solution tank mounted on waste-handling equipment to a first input of the nozzle. The compressed air may be supplied from a compressor tank mounted on waste-handling equipment to a second input of the nozzle. Flow of the compressed air from the compressor tank into the nozzle input may be regulated.

Embodiments of the disclosure are susceptible to being used for various purposes, including, though not limited to, enabling users to target a primary source of solid waste odors ensuring effective chemical distribution and the interaction of the dispersed chemical aerosol with odor-causing vapors emitted from the solid waste. Embodiments may be provided in mobile form as with disclosed waste-handling vehicles or may be offered as fixed-location systems.

Modifications to embodiments of the disclosure described in the foregoing are possible without departing from the scope of the disclosure as defined by the accompanying claims. Expressions such as “including”, “comprising”, “incorporating”, “consisting of”, “have”, “is” used to describe and claim the disclosure are intended to be construed in a non-exclusive manner, namely allowing for items, components or elements not explicitly described also to be present. Reference to the singular is also to be construed to relate to the plural.

What is claimed is:

1. An exposed waste odor neutralizing system, comprising:
mobile waste-handling equipment including at least one waste-handling effector;
an odor-mitigating solution tank mounted to the waste-handling equipment;
a compressor configured to pressurize ambient air; and
at least one nozzle configured to discharge a mixture of the pressurized ambient air and odor-mitigating solution from the odor-mitigating solution tank.

2. The system as set forth in claim 1, further comprising a compressor tank coupled with the compressor and configured to store the pressurized ambient air.

3. The system as set forth in claim 2, further comprising, coupled with the compressor tank, a flow regulator configured to control flow of pressurized air from the compressor tank.

4. The system as set forth in claim 1, further comprising a solution tank regulating pump configured to control flow of odor-mitigating solution from the odor-mitigating solution tank.

5. The system as set forth in claim 1, wherein the at least one nozzle further comprises:
an output;
a first input coupled with the odor-mitigating solution tank and the output; and
a second input coupled with the compressor and the output.

6. The system as set forth in claim 1, wherein the waste-handling equipment further includes at least one radiator fan.

7. The system as set forth in claim 6, wherein the at least one nozzle is further configured to discharge the mixture of the pressurized ambient air and odor-mitigating solution downstream of the radiator fan of the waste-handling equipment.

8. The system as set forth in claim 1, further comprising an odor-mitigating solution contained within the odor-mitigating solution tank.

9. An odor neutralizer dispersal system, comprising:
an odor-mitigating solution tank configured for mounting to mobile waste-handling equipment which includes at least one waste-handling blade or bucket;
a compressor configured for mounting to the waste-handling equipment and for pressurizing ambient air; and
at least one nozzle configured for mounting to the mobile waste-handling equipment and for discharging a mixture of the odor-mitigating solution and the pressurized ambient air.

10. The system as set forth in claim 9, further comprising a compressor tank configured for coupling with the compressor to contain the pressurized ambient air.

11. The system as set forth in claim 10, further comprising, a flow regulator configured for coupling with the compressor tank to control flow of pressurized ambient air therefrom.

12. The system as set forth in claim 9, further comprising a solution tank regulating pump configured for coupling with the odor-mitigating solution tank to control flow of odor-mitigating solution therefrom.

13. The system as set forth in claim 9, wherein the at least one nozzle further comprises:
an output configured to emit a mixture of odor-mitigating solution;
in fluid communication with the output, a first input configured for coupling with the odor-mitigating solution tank; and
in fluid communication with the output, a second input configured for coupling with the compressor.

14. The system as set forth in claim 9, wherein the waste-handling equipment includes at least one radiator fan.

15. The system as set forth in claim 14, wherein the at least one nozzle is further configured for mounting downstream of the radiator fan.

16. A method for mitigating waste-handling facility odor, comprising:
at the working area of a waste-handling facility, manipulating waste with a blade or bucket of a mobile waste-handling machine; and
contemporaneously with the manipulating, discharging a mixture of odor-mitigating solution and compressed air.

17. The method as set forth in claim 15, wherein discharging the mixture further comprises discharging through an output of a nozzle.

18. The method as set forth in claim 15, wherein the manipulating waste further comprises manipulating with waste-handling equipment having at least one radiator fan with rotating vanes.

19. The method as set forth in claim 18, wherein discharging a mixture of odor-mitigating solution further comprises discharging the mixture downstream of the radiator fan rotating vanes.

20. The method as set forth in claim 15, further comprising regulating the pressure of the compressed air from the compressor tank.

21. The method as set forth in claim 15, further comprising pumping the odor-mitigating solution from an odor-mitigating solution tank mounted on the waste-handling equipment to a first input of the nozzle.

22. The method as set forth in claim 21, further comprising pumping the compressed air from a compressor tank mounted on the waste-handling equipment to a second input of the nozzle.
